(19) United States
(12) Patent Application Publication
(10) Pub. No.: US 2007/0270399 A1
(43) Pub. Date: Nov. 22, 2007

(54) New compounds 302

(76) Inventors: Marlene Fredenwall, Molndal (SE); Anders Johansson, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 11/749,951

(22) Filed: May 17, 2007

Related U.S. Application Data
(60) Provisional application No. 60/801,578, filed on May 18, 2006.

(51) Int. Cl. A61K 31/5377; C07D 413/04

(52) U.S. Cl. 514/210.2; 544/111

(57) ABSTRACT

The present invention relates to new compounds of formula I, to pharmaceutical compositions comprising said compounds, and to the use of said compounds in therapy. The present invention further relates to processes for the preparation of compounds of formula I.

This application claims the priority benefit of U.S Provisional Application No. 60/801,578, filed May 18, 2006.

### FIELD OF THE INVENTION

The present invention relates to new compounds of formula I, to pharmaceutical compositions comprising said compounds, and to the use of said compounds in therapy. The present invention further relates to processes for the preparation of compounds of formula I.

### BACKGROUND OF THE INVENTION

The neurokinins, also known as the tachykinins, comprise a class of peptide neurotransmitters which are found in the peripheral and central nervous systems. The three principal tachykinins are Substance P (SP), Neurokinin A (NKA) and Neurokinin B (NKB). At least three receptor types are known for the three principal tachykinins. Based upon their relative selectivities favouring the agonists SP, NKA and NKB, the receptors are classified as neurokinin 1 (NK₁), neurokinin 2 (NK2) and neurokinin 3 (NK₃) receptors, respectively.

There is a need for an orally active NK receptor antagonist for the treatment of e.g. respiratory, cardiovascular, neuro, pain, oncology, inflammatory and/or gastrointestinal disorders. In order to increase the therapeutic index of such therapy it is desirable to obtain such a compound possessing no or minimal toxicity as well as being selective to said NK receptors. Furthermore, it is considered necessary that said medicament has favourable pharmacokinetic and metabolic properties thus providing an improved therapeutic and safety profile such as lower liver enzyme inhibiting properties.

It is well known that certain compounds may cause undesirable effects on cardiac repolarisation in man, observed as a prolongation of the QT interval on electrocardiograms (ECG). In extreme circumstances, this drug-induced prolongation of the QT interval can lead to a type of cardiac arrhythmia called Torsades de Pointes (TdP; Vandenberg et al. hERG K⁺ channels: friend and foe. Trends Pharmacol Sci 2001; 22: 240-246), leading ultimately to ventricular fibrillation and sudden death. The primary event in this syndrome is inhibition of the rapid component of the delayed rectifying potassium current (IKr) by these compounds. The compounds bind to the aperture-forming alpha sub-units of the channel protein carrying this current. The aperture-forming alpha sub-units are encoded by the human ether-a-go-go-related gene (hERG). Since IKr plays a key role in repolarisation of the cardiac action potential, its inhibition slows repolarisation and this is manifested as a prolongation of the QT interval. Whilst QT interval prolongation is not a safety concern per se, it carries a risk of cardiovascular adverse effects and in a small percentage of people it can lead to TdP and degeneration into ventricular fibrillation.

In particular, it is desirable that the NK receptor antagonist has a suitable balance of pharmacodynamic and pharmacokinetic properties to make it therapeutically useful. In addition to having sufficient and selective potency, the NK receptor antagonist needs to be balanced with regard to relevant pharmacokinetic properties. Thus, it is necessary that the NK antagonist has: a) sufficiently high affinities at the different NK receptors, b) pharmacokinetic properties (absorption, distribution and elimination properties) that makes it possible for the drug to act at the targeted NK receptors in the periphery as well as in the CNS. For instance, the NK receptor antagonist needs to have sufficiently high metabolic stability, c) sufficiently low affinities to different ion channels, such as the hERG-encoded potassium channel in order to obtain a tolerable safety profile and d) liver enzyme (such as CYP3A4) inhibiting properties at a low level to prevent drug-drug interactions.

Furthermore, in order to enhance the efficacy of the NK receptor antagonist, it is beneficial to have an NK antagonist with a long-lasting competitive mode of action at the receptor.

EP 0625509, EP 0630887, WO 95/05377, WO 95/12577, WO 95/15961, WO 96/24582, WO 00/02859, WO 00/20003, WO 00/20389, WO 00/25766, WO 00/34243, WO 02/51807 and WO 03/037889 disclose piperidinylbutylamide derivatives, which are tachykinin antagonists.

“4-Amino-2-(aryl)-butylbenzamides and Their Conformationally Constrained Analogues. Potent Antagonists of the Human Neurokinin-2 (NK₂) Receptor”, Roderick MacKenzie, A., et al, Bioorganic & Medicinal Chemistry Letters (2003), 13, 2211-2215, discloses the compound N-[2-(3,4-dichlorophenyl)-4-(3-morpholin-4-ylazetidin-1-yl)butyl]-N-methylbenzamide which was found to possess functional NK₂ receptor antagonistic properties.

WO 96/05193, WO 97/27185 and EP 0962457 disclose azetidinylalkyllactam derivatives with tachykinin antagonist activity.

EP 0790248 discloses azetidinylalkylazapiperidones and azetidinylalkyloxapiperidones, which are stated to be tachykinin antagonists.

WO 99/01451 and WO 97/25322 disclose azetidinylalkylpiperidine derivatives claimed to be tachykinin antagonists.

EP 0791592 discloses azetidinylalkylglutarimides with tachykinin antagonistic properties.

WO2004/110344 A2 discloses dual NK1,2 antagonists and the use thereof.

An object of the present invention was to provide novel neurokinin antagonists useful in therapy. A further object was to provide novel compounds having well-balanced pharmacokinetic and pharmacodynamic properties.

### OUTLINE OF THE INVENTION

The present invention provides a compound of the general formula (I)

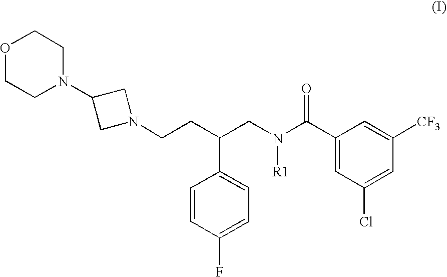

wherein

R1 is C₁-C₄ alkyl, wherein one or more of the hydrogen atoms of the alkyl group may be substituted for a fluoro atom;

as well as pharmaceutically and pharmacologically acceptable salts thereof, and enantiomers of the compound of formula I and salts thereof.

The present invention relates to compounds of formula I as defined above as well as to salts thereof. Salts for use in pharmaceutical compositions will be pharmaceutically acceptable salts, but other salts may be useful in the production of the compounds of formula I.

The compounds of the present invention are capable of forming salts with various inorganic and organic acids and such salts are also within the scope of this invention. Examples of such acid addition salts include acetate, adipate, ascorbate, benzoate, benzenesulfonate, bisulfate, butyrate, camphorate, camphorsulfonate, citrate, cyclohexyl sulfamate, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, 2-naphthalenesulfonate, nitrate, oxalate, palmoate, persulfate, phenylacetate, phosphate, picrate, pivalate, propionate, quinate, salicylate, stearate, succinate, sulfamate, sulfanilate, sulfate, tartrate, tosylate (p-toluenesulfonate), and undecanoate.

Pharmaceutically acceptable salts may be prepared from the corresponding acid in conventional manner. Non-pharmaceutically-acceptable salts may be useful as intermediates and as such are another aspect of the present invention.

Acid addition salts may also be in the form of polymeric salts such as polymeric sulfonates.

The salts may be formed by conventional means, such as by reacting the free base form of the product with one or more equivalents of the appropriate acid in a solvent or medium in which the salt is poorly soluble, or in a solvent such as water, which is removed in vacuo or by freeze drying or by exchanging the anions of an existing salt for another anion on a suitable ion-exchange resin.

Compounds of formula I have one or more chiral centres, and it is to be understood that the invention encompasses all optical isomers, enantiomers and diastereomers. The compounds according to formula (I) can be in the form of the single stereoisomers, i.e. the single enantiomer (the R-enantiomer or the S-enantiomer) and/or diastereomer. The compounds according to formula (I) can also be in the form of a racemic mixture, i.e. an equimolar mixture of enantiomers.

The compounds can exist as a mixture of conformational isomers. The compounds of this invention comprise both mixtures of, and individual, conformational isomers.

As used herein, the term “C₁-C₄ alkyl” includes straight as well as branched chain C₁₋₄ alkyl groups, for example methyl, ethyl, n-propyl, i-propyl, n-butyl, i-butyl, s-butyl or t-butyl.

### Pharmaceutical Formulations

According to one aspect of the present invention there is provided a pharmaceutical formulation comprising a compound of formula I, as a single enantiomer, a racemate or a mixture thereof as a free base or pharmaceutically acceptable salts thereof, for use in prevention and/or treatment of respiratory, cardiovascular, neuro, pain, oncology, inflammatory and/or gastrointestinal disorders.

The pharmaceutical compositions of this invention may be administered in standard manner for the disease condition that it is desired to treat, for example by oral, topical, parenteral, buccal, nasal, vaginal or rectal administration or by inhalation or insufflation. For these purposes the compounds of this invention may be formulated by means known in the art into the form of, for example, tablets, pellets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

In addition to the compounds of the present invention the pharmaceutical composition of this invention may also contain, or be co-administered (simultaneously or sequentially) with, one or more pharmacological agents of value in treating one or more disease conditions referred to herein.

The pharmaceutical compositions of this invention will normally be administered to humans in a daily dose of a compound of formula I of from 0.01 to 25 mg/kg body weight. Alternatively, a daily dose of the compound of formula I from 0.1 to 5 mg/kg body weight is administered. This daily dose may be given in divided doses as necessary, the precise amount of the compound administered and the route of administration depending on the weight, age and sex of the patient being treated and on the particular disease condition being treated according to principles known in the art.

Typically unit dosage forms will contain about 1 mg to 500 mg of a compound of this invention. For example a tablet or capsule for oral administration may conveniently contain up to 250 mg (and typically 5 to 100 mg) of a compound of the formula (I) or a pharmaceutically acceptable salt thereof. In another example, for administration by inhalation, a compound of the formula (I) or a pharmaceutically acceptable salt thereof may be administered in a daily dosage range of from 5 to 100 mg, in a single dose or divided into two to four daily doses. In a further example, for administration by intravenous or intramuscular injection or infusion, a sterile solution or suspension containing up to 10% w/w (and typically 5% w/w) of a compound of the formula (I) or a pharmaceutically acceptable salt thereof may be used.

### Medical and Pharmaceutical Use

The present invention provides a method of treating or preventing a disease condition wherein antagonism of tachykinins acting at the NK receptors is beneficial which comprises administering to a subject an effective amount of a compound of the formula (I) or a pharmaceutically-acceptable salt thereof. The present invention also provides the use of a compound of the formula (I) or a pharmaceutically acceptable salt thereof in the preparation of a medicament for use in a disease condition wherein antagonism of tachykinins acting at the NK receptors is beneficial.

The compounds of formula (I) or pharmaceutically acceptable salts or solvates thereof may be used in the manufacture of a medicament for use in the prevention or treatment of respiratory, cardiovascular, neuro, pain, oncology and/or gastrointestinal disorders.

Examples of such disorders are asthma, allergic rhinitis, pulmonary diseases, cough, cold, inflammation, chronic obstructive pulmonary disease, airway reactivity, urticaria, hypertension, rheumatoid arthritis, edema, angiogenesis, pain, migraine, tension headache, psychoses, depression, anxiety, Alzheimer's disease, schizophrenia, Huntington's disease, bladder hypermotility, urinary incontinence, eating disorder, manic depression, substance dependence, movement disorder, cognitive disorder, obesity, stress disorders, micturition disorders, mania, hypomania and aggression, bipolar disorder, cancer, carcinoma, fibromyalgia, non cardiac chest pain, gastrointestinal hypermotility, gastric asthma, Crohn's disease, gastric emptying disorders, ulcerative colitis, irritable bowel syndrome (IBS), inflammatory bowel disease (IBD), emesis, gastric asthma, gastric motility disorders, gastro-esophageal reflux disease (GERD) or functional dyspepsia.

### Methods of Preparation

In another aspect the present invention provides a process for preparing a compound of the formula (I) or salts thereof which process comprises:

a) reacting a compound of the formula (II) with a compound of the formula (III):

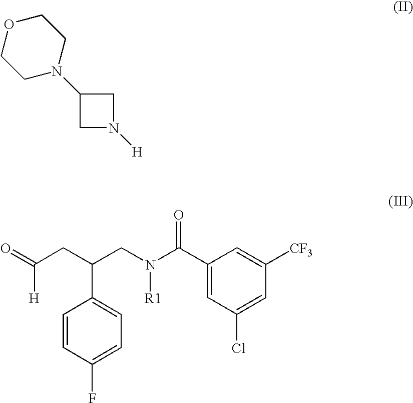

wherein R1 is as hereinbefore defined; and the conditions are such that reductive alkylation of the compound of the formula (II) forms an N—C bond between the nitrogen atom of the azetidine group of the compound of formula (II) and the carbon atom of the aldehyde group of the compounds of formula (III); or

b) reacting a compound of the formula (II) with a compound of the formula (IV):

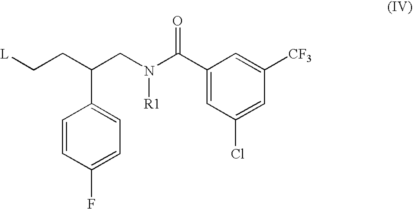

wherein R1 is as hereinbefore defined; and L is a group such that alkylation of the compound of the formula (II) forms an N—C bond between the nitrogen atom of the azetidine group of the compound of formula (II) and the carbon atom of the compounds of formula (IV) that is adjacent to the L group; or

c) reacting a compound of the formula (V) with a compound of the formula (VI):

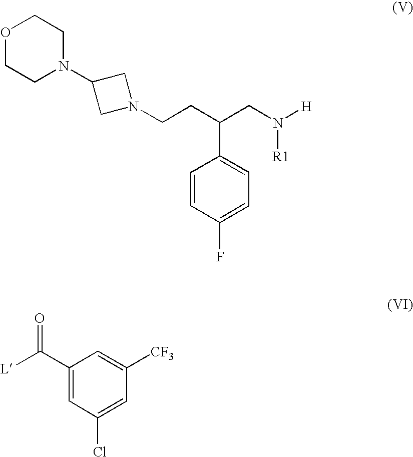

wherein R1 is as hereinbefore defined; and L′ is a leaving group;

and optionally forming a pharmaceutically acceptable salt.

The compounds of the formulae (II) and (III) are reacted under conditions of reductive alkylation. The reaction is typically performed at a non-extreme temperature, for example 0-40 ° C., in a substantially inert solvent for example dichloromethane. Typical reducing agents include borohydrides such as sodium cyanoborohydride.

The compounds of the formulae (II) and (IV) are reacted under conditions of alkylation. Typically in the compounds of the formula (IV) L is a leaving group such as halogen or alkylsulfonyloxy. The reaction is typically performed at an elevated temperature, for example 30-130 ° C., in a substantially inert solvent for example DMF.

The compound of the formula (II) is known in the art. Its synthesis is described as for instance in WO 00/63168. The compounds of the formula (III) may be prepared, for example, by reacting a compound of the formula (VI) with a compound of the formula (VII):

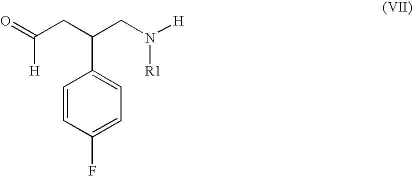

wherein R1 is as hereinbefore defined under conventional acylation conditions.

The compounds of the formula (IV) may be prepared, for example, by reacting a compound of the formula (VI) with a compound of the formula (VIII):

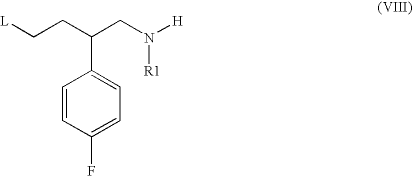

wherein R1 is as hereinbefore defined under conventional acylation conditions.

The compounds of the formulae (V) and (VI) may be reacted under conventional acylation conditions wherein

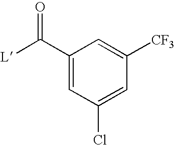

is an acid or an activated acid derivative. Such activated acid derivatives are well known in the literature. They may be formed in situ from the acid or they may be prepared, isolated and subsequently reacted. Typically L′ is chloro thereby forming the acid chloride. Typically the acylation reaction is performed in the presence of a non-nucleophilic base, for example N,N-diisopropylethylamine, in a substantially inert solvent such as dichloromethane at a non-extreme temperature.

The compounds of the formula (VII) and (VIII) are known or may be prepared in conventional manner.

### EXAMPLES

It should be emphasised that the compounds of the present invention most often show highly complex NMR spectra due to the existence of conformational isomers. This is believed to be a result from slow rotation about the amide and/or aryl bond. The following abbreviations are used in the presentation of the NMR data of the compounds: s-singlet; d-doublet; t-triplet; qt-quartet; qn-quintet; m-multiplet; b-broad; cm-complex multiplet, which may include broad peaks.

The following examples will describe, but not limit, the invention.

The following abbreviations are used in the experimental: DMSO (dimethylsulfoxide), THF (tetrahydrofuran), MTBE (methyl tert-butyl ether) and RT (room temperature).

### Example 1

### 3-Chloro-N-[(2S)-2-(4-fluorophenyl)-4-(3-morpholin-4-ylazetidin-1-yl)butyl]-N-methyl-5-(trifluoromethyl)benzamide

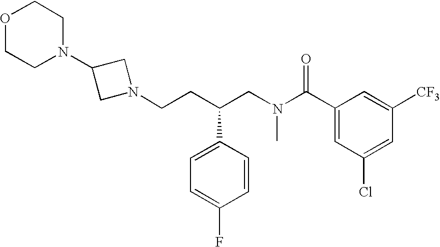

A mixture of 3-chloro-N-[(2S)-2-(4-fluorophenyl)-4-oxobutyl]-N-methyl-5-(trifluoromethyl)benzamide (see method 1; 0.25 g, 0.62 mmol), 4-azetidin-3-ylmorpholine hydrochloride (see WO 00/63168; 0.17 g, 0.93 mmol), triethylamine (0.13 g, 1.24 mmol) and methanol (7 mL) was stirred at RT for 30 minutes. Sodium triacetoxyborohydride (0.26 g, 1.24 mmol) was added and the mixture was stirred at RT for 2 h. The solvent was removed by evaporation. The residue was dissolved in methylene chloride and the solution was washed with aqueous NaHCO₃. The organic phase was separated and the solvent was removed by evaporation. The product was purified by chromatography on silica gel (ammonia saturated methanol-methylene chloride, 2% to 15% MeOH). There was obtained 0.23 mg (69%) of the title compound as a colourless oil. ¹H NMR (500 MHz, CD₃OD): δ 1.3-3.8 (cm, 23H), 6.7-7.2 (cm, 6H), 7.5 (s, 1H); LCMS: m/z 528 (M+1)⁺.

### Preparation of Starting Materials

The starting materials for the example above are either commercially available or are readily prepared by standard methods from known materials. For example, the following reactions are an illustration, but not a limitation, of some of the starting materials.

### Method 1

### 3-Chloro-N-[(2S)-2-(4-fluorophenyl)-4-oxobutyl-N-methyl-5-(trifluoromethyl)benzamide

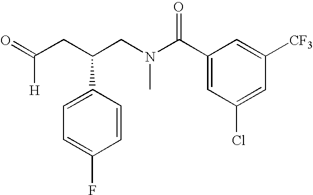

### (a) tert-Butyl 3-cyano-3-(4-fluorophenyl)propanoate

Lithium diisopropylamide (LDA, 52 L, 1.8 M, 93.6 mol) in a solution of THF/heptane and ethylbenzene was charged to a reactor under a nitrogen atmosphere, and THF (52 L) was then added. The temperature was adjusted to an inner temperature (the temperature of the reaction solution) of −48° C. 4-Fluorophenylacetonitrile (13.0 kg, 96.2 mol) in a THF-solution (25 L) was charged during 1 h and 50 min to the solution comprising LDA, while the temperature of the reaction mixture was kept below −30° C. The temperature was increased to −6° C. over 1 h, during that time the yellow slurry transformed into a dark purple solution. THF (5 L) followed by tert-butylbromoacetate (20.25 kg, 104 mol) and finally THF (25 L) were charged to a second reactor. The temperature was lowered to an inner temperature of −48° C. The dark purple solution above was charged to the tert-butyl-bromoacetate-solution over 7.5 h, while the inner temperature was kept below −34° C. The inner temperature was adjusted to −5° C. and the reaction mixture was quenched by adding a solution of ammonium chloride (12.7 kg) and water (55 L) over 15 min. Methyl tert-butyl ether (43 L) was charged and the obtained mixture was stirred for 5 min. After phase separation, the aqueous phase was discarded. Brine (7.6 kg sodium chloride in 25 L of water) was charged to the remaining organic phase and the mixture was stirred for 5 min. The aqueous phase was discarded and the remaining solution was concentrated by distillation at reduced pressure to a volume of 150 L. Isooctane (43 L) was charged and the distillation was continued until the resulting volume was 60 L at which point crystallization started. MTBE (25 L) was charged and the jacket temperature was set to 0° C. After 2 h the batch was filtered (inner temperature 2° C.) and washed with isooctane (2×20 L). After drying 16.8 Kg (72%) of tert-butyl 3-cyano-3-(4-fluorophenyl)propanoate was obtained. ¹H NMR (DMSO-d₆) δ 7.51 (app d, J=8 Hz, 1 H), 7.50 (app d, J=8 Hz, 1 H), 7.24 (app t, J=8 Hz, 2H), 4.50 (app dd, J₁=6 Hz, J₂=8 Hz, 1 H), 3.02 (app dd, J₁=8 Hz, J₂=16 Hz, 1 H), 2.86 (app dd, J₁=6 Hz, J₂=16 Hz, 1 H), 1.36 (s, 9 H); ¹³C NMR (DMSO-d₆) δ 168.4, 161.7 (d, J_{C,F}=244 Hz), 131.3 (d, J_{C,F}=3 Hz), 129.8 (d, J_{C,F}=9 Hz), 120.6, 115.7 (d, J_{C,F}=22 Hz), 81.0, 39.1, 31.4, 27.6.

### (b) 4-Amino-3-(4-fluorophenyl)butan-1-ol

tert-Butyl 3-cyano-3-(4-fluorophenyl)propanoate (16.7 kg, 67.0 mol) was charged under nitrogen atmosphere to a reactor and THF (50 L) was then added. The temperature was adjusted to an inner temperature of 65° C. Borane-dimethylsulfide complex (16.6 L, 166 mol) in a THF solution (5 L) was charged to the reaction mixture over a period of 43 minutes. The mixture was then refluxed for 2 hours. The reaction mixture was cooled to 10° C. Water (75 L) and hydrochloric acid (25.5 L) was charged to a second vessel and the reaction solution above was charged to this aqueous phase accompanied by gas evolution (H₂ is formed). When the addition was complete (after 1.5 h), the jacket temperature was increased to 105° C. and the solvents were distilled off until the temperature of the reaction mixture reached 85° C. The reaction mixture was refluxed for 12.5 h and then cooled to 24° C. Aqueous sodium hydroxide (50% solution, 32.4 kg) was charged followed by toluene (55 L) and THF (18 L). After phase separation, the aqueous phase was extracted with a mixture of toluene (30 L) and THF (13 L). The organic phases were combined and approximately 65 L of solvent mixture was removed by distillation under reduced pressure. Toluene (40 L) and THF (5 L) was charged to the organic phase and the resulting mixture was clear filtered and returned to the reactor. The solvents were distilled off at reduced pressure until 50 L remained. Toluene (20 L) was charged and the distillation was continued until approximately 35 L remained. The inner temperature was lowered from 59° C. to 12° C. over 1 h and seeding crystals (0.2 g) were added, which started the crystallization. Heptane (12 L) was charged and the slurry was cooled down to 6° C. over 2 h. The slurry was filtered and the solid was washed with heptane (2×10 L) and dried. There was obtained 6.13 kg (50%) of 4-amino-3-(4-fluorophenyl)butan-1-ol. ¹H NMR (DMSO-d₆) δ 7.21 (app d, J=8 Hz, 1 H), 7.19 (app d, J=8 Hz, 1 H), 7.10 (app t, J=8 Hz, 2H), 3.13-3.35 (m, 2 H), 2.59-2.81 (m, 2 H), 1.77-1.94 (m, 2 H), 1.50-1.68 (m, 2 H); ¹³C NMR (CDCl₃) δ 161.7 (d, J_{C,F}=244 Hz), 139.9 (d, J_{C,F}=3 Hz), 129.0 (d, J_{C,F}=8 Hz), 115.6 (d, J_{C,F}=21 Hz), 61.1, 48.2, 46.7, 38.6.

### (c) (3S)-4-Amino-3-(4-fluorophenyl)butan-1-ol (R)—O-acetylmandelic acid salt

(R)—O-Acetylmandelic acid (18.79 kg, 96.76 mol) was charged to a reactor followed by water (845 g) and ethyl acetate (100 L). The solution was stirred at an inner temperature of 17-20° C. for 0.5 h. The clear solution was collected in a drum and the reactor was rinsed with ethyl acetate (20 L). The rinsing solution was then combined with the above clear (R)—O-acetylmandelic acid solution. 4-Amino-3-(4-fluoro-phenyl)-butan-1-ol (20.64 kg, 112.65 mol) was charged to a reactor followed by absolute ethanol (99.7% w/w, 19 L) and ethyl acetate (43 L). Stirring was started and the inner temperature was raised to 59° C. The (R)—O-acetylmandelic acid solution was charged to the solution of 4-amino-3-(4-fluoro-phenyl)-butan-1-ol over 24 min. The dark yellow solution thus obtained started to crystallize at an inner temperature of 53° C. about 5 min after complete addition of (R)—O-acetylmandelic acid. The inner temperature was kept at 52-53° C. for 20 min, and the slurry was then cooled down to 25° C. over 1 h and 20 min. The white slurry was filtered and the solid was washed with ethyl acetate (2×37.5 L) to give, after drying on the filter, 15.33 kg of needle like white crystals having an optical purity of 83% enantiomeric excess (ee). The ee corrected yield is 66%. The obtained product (15.33 kg, 40.62 mol) was charged to a reactor followed by absolute 99.5% ethanol (27.5 L) and ethyl acetate (22.5 L). Stirring was started and the mixture was heated to an inner temperature of 70° C. Ethyl acetate (105 L) was charged to the mixture over 44 min. The inner temperature was kept between 67-70° C. during the addition. The crystallization started 8 min after the last addition of ethyl acetate (inner temperature 69° C.). The slurry was cooled to an inner temperature of 25° C. over 1 h and 50 min and then filtered. The solid was washed with ethyl acetate (2×37.5 L) and dried giving 11.65 kg (82% ee corrected yield) of (3S)-4-amino-3-(4-fluorophenyl)butan-1-ol as white crystals. The optical purity was 98% ee according to chiral HPLC. ¹H NMR (DMSO-d₆) δ 7.41 (app dd, J₁=7 Hz, J₂=1 Hz, 2 H), 7.16-7.34 (m, 5 H), 7.12 (app t, J=9 Hz, 2H), 5.53 (app s, 1 H), 3.08-3.33 (m, 2 H), 2.92-3.08 (m, 2 H), 2.78-2.92 (m, 1 H), 2.04 (s, 3 H), 1.77-1.94 (m, 1 H), 1.50-1.69 (m, 1 H); ¹³C NMR (DMSO-d₆) δ 170.6, 169.7, 168.4, 161.1 (d, J_{C,F}=242 Hz), 138.3, 137.7 (d, J_{C,F}=3 Hz), 129.7 (d, J_{C,F}=8 Hz), 127.9, 127.4, 127.3, 115.2 (d, J_{C,F}=21 Hz), 77.2, 58.2, 44.0, 38.7, 36.3, 21.1. [α]_{D} (c 1.0 in methanol, 25° C.) −60.4°.

### (d) Ethyl [(2S)-2-(4-fluorophenyl)-4-hydroxybutyl]carbamate

(S)-4-Amino-3-(4-fluorophenyl)-butan-1-ol (R)—O-acetylmandelic acid salt (11.61 kg, 30.76 mol) was charged to a stirred solution of aqueous sodium hydroxide (11.30 kg of 50% sodium hydroxide in water, 141.3 mol, diluted to approximately 70 L) at 16° C. inner temperature under nitrogen atmosphere. THF (7.5 L) and toluene (74 L) was charged resulting in a clear two-phase system. The solution was cooled to −1° C. and ethyl chloroformate (3.60 kg, 33.2 mol) in a mixture of THF (1.1 L) and toluene (10 L) was charged to the mixture over 18 min. During the addition the inner temperature rose to 9° C. The reaction mixture was heated to 18° C. over 1 h and 48 min at which point HPLC analysis indicated that the reaction was complete. Toluene (17.5 L) was charged and good mixing was achieved followed by phase separation. The resulting two phases were separated and the aqueous phase was discarded. The organic phase was washed with water (3×8 L) and concentrated to approximately 50 L by distillation at reduced pressure. Toluene (25 L) was charged and the distillation was continued until approximately 30 L of the solvents had been distilled off. Toluene (25 L) was charged and the distillation continued until approximately 40 L remained in. The toluene solution containing ethyl [(2S)-2-(4-fluorophenyl)-4-hydroxybutyl]carbamate was taken straight into the next step.

### (e) (3S)-3-(4-Fluorophenyl)-4-(methylamino)butan-1-ol

Lithium aluminium hydride (2.11 kg, 55.6 mol) was charged to a reactor containing THF (50 L) at an inner temperature of 20° C. under a nitrogen atmosphere, while stirring. The mixture was heated to an inner temperature of 51° C. and ethyl [(2S)-2-(4-fluorophenyl)-4-hydroxybutyl]carbamate in toluene (total volume 43 L) from the previous step was charged to the lithium aluminium hydride slurry in THF over 2 h. The temperature was kept between 51-68° C. during the addition. The charging vessel was rinsed with toluene (5 L) and the batch was then held at 56-58° C. for 2 h. The reaction mixture was cooled to an inner temperature of 2° C. and a solution of aqueous sodium bicarbonate (26 L) was charged over 44 min (inner temperature 15° C. and jacket temperature −25° C. at the end of the quench) after which the jacket was adjusted to 20° C. and the batch was left for 15 h. The slurry in the reactor was filtered and the resulting solid was washed with toluene (30 L) in four portions. The filtrate was returned to the reactor (cleaned from aluminium salts) and washed with water (2×10 L) and then clear filtered. The clear filtered solution was returned to the reactor and concentrated to approximately 15 L by distillation under reduced pressure. The distillation was stopped and isooctane (30 L) was charged to the slurry. The slurry was cooled from an inner temperature of 32° C. to 20° C. over 40 min, then filtered and the isolated solid was washed with isooctane (30 L) in four portions. The solid was dried and this resulted in 4.54 kg (75% over two Steps) of (3S)-3-(4-fluorophenyl)-4-(methylamino)butan-1-ol. ¹H NMR (DMSO-d₆) δ 7.22 (app d, J=8 Hz, 1 H), 7.20 (app d, J=8 Hz, 1 H), 7.08 (app t, J=8 Hz, 2H), 3.11-3.34 (m, 2 H), 3.72-3.88 (m, 1 H), 3.52-3.66 (m, 2 H), 2.21 (s, 3 H), 1.73-1.91 (m, 1 H), 1.48-1.68 (m, 1 H); ¹³C NMR δ 160.6 (d, J_{C,F}=241 Hz), 140.7 (d, J_{C,F}=3 Hz), 129.3 (d, J_{C,F}=8 Hz), 114.8 (d, J_{C,F}=21 Hz), 58.9, 57.8, 41.3, 37.4, 36.1. [α]_{D} (c 1.0 in methanol, 25° C.) +8.8°.

### (f) 3-Chloro-N-[(2S)-2-(4-fluorophenyl)-4-hydroxybutyl]-N-methyl-5-(trifluoromethyl)benzamide

(3S)-3-(4-Fluorophenyl)-4-(methylamino)butan-1-ol (4.0 g, 20.5 mmol) was mixed with an aqueous solution of NaOH (3.3 g in 16 mL of water, 41 mmol). To the formed suspension was added by drops a toluene solution of 3-chloro-5-(trifluoromethyl)benzoyl chloride (5.0 g in 24 mL of toluene, 20.5 mmol) while vigorously stirring. The addition was completed after 15 min. The mixture was stirred for 1 h at RT. The aqueous phase was separated off and the organic solution was washed twice with water. The solvent was dried and then removed by evaporation. There was obtained 8.6 g (100%) of 3-chloro-N-[(2S)-2-(4-fluorophenyl)-4-hydroxybutyl]-N-methyl-5-(trifluoromethyl)benzamide as a viscous oil. ¹H NMR (500 MHz, CDCl₃): 1.6-2.0 (cm, 2H), 2.7 (s, 3H), 3.0-3.8 (cm, 5H), 6.8-7.3 (cm, 6H), 7.6 (s, 1H); LCMS: m/z 404 (M+1)⁺.

### (g) 3-Chloro-N-[(2S)-2-(4-fluorophenyl)-4-oxobutyl]-N-methyl-5-(trifluoromethyl)benzamide

3-Chloro-N-[(2S)-2-(4-fluorophenyl)-4-hydroxybutyl]-N-methyl-5-(trifluoromethyl)benzamide (8.5 g, 21.0 mmol) was dissolved in DMSO (30 mL) together with triethylamine (8.5 g, 84.2 mmol). Sulfur trioxide pyridine complex (7.4 g, 46.3 mmol) dissolved in DMSO (30 mL) was added by drops over a period of 20 min. The mixture was stirred at RT for 3 h and then another portion of sulfur trioxide pyridine complex (3.5 g, 21.0 mmol) was added. The mixture was stirred at RT overnight and then concentrated on a rotavapor for 2 h in order to remove formed dimethylsulfide. The mixture was diluted with MTBE (50 mL) and then sulfuric acid (2.0 g, 21.0 mmol) dissolved in water (40 mL) was added by drops. The mixture was stirred vigorously for 25 min, the two phases were separated and then the organic solution was washed twice with water. The solution was dried and the solvent was removed by evaporation. The product was purified by chromatography on silica gel (ethyl acetate-heptane, 10% to 100% ethyl acetate). There was obtained 2.6 g (30%) of the title compound as an oil. ¹H NMR (500 MHz, CDCl₃): δ 2.6-3.9 (cm, 8H), 6.8-7.4 (cm, 6H), 7.6 (d, 1H), 9.6-9.8 (d, 1H); LCMS: m/z 400 (M−1)⁺.

### Pharmacology

### Transfection and Culturing of Cells Used in FLIPR and Binding Assays

Chinese Hamster Ovary (CHO) K1 cells (obtained from ATCC) were stably transfected with the human NK₂ receptor (hNK₂R cDNA in pRc/CMV, Invitrogen) or the human NK₃ receptor (hNK₃R in pcDNA 3.1/Hygro (+)/IRES/CD8, Invitrogen vector modified at AstraZeneca EST-Bio UK, Alderley Park). The cells were transfected with the cationic lipid reagent LIPOFECTAMINE™ (Invitrogen) and selection was performed with Geneticin (G418, Invitrogen) at 1 mg/ml for the hNK₂R transfected cells and with Hygromycin (Invitrogen) at 500 μg/ml for the hNK₃R transfected cells. Single cell clones were collected by aid of Fluorescence Activated Cell Sorter (FACS), tested for functionality in a FLIPR assay (see below), expanded in culture and cryopreserved for future use. CHO cells stably transfected with human NK₁ receptors originates from AstraZeneca R&D, Wilmington USA. Human NK₁ receptor cDNA (obtained from RNA-PCR from lung tissue) was subcloned into pRcCMV (Invitrogen). Transfection was performed by Calcium Phosphate and selection with 1 mg/ml G418.

The CHO cells stably transfected with hNK₁R, hNK₂R and hNK₃R were cultured in a humidified incubator under 5% CO₂, in Nut Mix F12 (HAM) with Glutamax I, 10% Foetal Bovine Serum (FBS), 1% Penicillin/Streptomycin (PEST) supplemented with 200 μg/ml Geneticin for the hNK₁R and hNK₂R expressing cells and 500 μg/ml Hygromycin for the hNK₃R expressing cells. The cells were grown in T175 flasks and routinely passaged when 70-80% confluent for up to 20-25 passages.

### Assessing the Activity of Selected test Compounds to Inhibit Human NK₁/NK₂NK₃ Receptor Activation (FLIPR Assay)

The activity of a compound of the invention to inhibit NK₁/NK₂/NK₃ receptor activation measured as NK₁/NK₂/NK₃ receptor mediated increase in intracellular Ca²⁺ was assessed by the following procedure:

CHO cells stably transfected with human NK₁, NK₂ or NK₃ receptors were plated in black walled/clear bottomed 96-well plates (Costar 3904) at 3.5×10⁴ cells per well and grown for approximately 24 h in normal growth media in a 37° C. CO₂-incubator.

Before the FLIPR assay the cells of each 96-well plate were loaded with the Ca²⁺ sensitive dye Fluo-3 (TEFLABS 0116) at 4 μM in a loading media consisting of Nut Mix F12 (HAM) with Glutamax I, 22 mM HEPES, 2.5 mM Probenicid (Sigma P-8761) and 0.04% Pluronic F-127 (Sigma P-2443) for 1 h kept dark in a 37° C. CO₂-incubator. The cells were then washed three times in assay buffer (Hanks balanced salt solution (HBSS) containing 20 mM HEPES, 2.5 mM Probenicid and 0.1% BSA) using a multi-channel pipette leaving them in 150 μl at the end of the last wash. Serial dilutions of a test compound in assay buffer (final DMSO concentration kept below 1%) were automatically pipetted by FLIPR (Fluorometric Imaging Plate Reader) into each test well and the fluorescence intensity was recorded (excitation 488 nm and emission 530 nm) by the FLIPR CCD camera for a 2 min pre-incubation period. 50 μl of the Substance P (NK₁ specific), NKA (NK₂ specific), or Pro-7-NKB (NK₃ specific) agonist solution (final concentration equivalent to an approximate EC₆₀ concentration) was then added by FLIPR into each well already containing 200 μl assay buffer (containing the test compound or vehicle) and the fluorescence was continuously monitored for another 2 min. The response was measured as the peak relative fluorescence after agonist addition and IC₅₀s were calculated from ten-point concentration-response curves for each compound. The IC₅₀s were then converted to pK_{B} values with the following formula:

K_{B}=IC₅₀/1+(EC₆₀ conc. of agonist used in assay/EC₅₀ agonist)

pK_{B}=−log K_{B}

### Determining the Dissociation Constant (Ki) of Compounds for Human NK₁/NK₂/NK₃ Receptors (Binding Assay)

Membranes were prepared from CHO cells stably transfected with human NK₁, NK₂ or NK₃ receptors according to the following method.

Cells were detached with Accutase® solution, harvested in PBS containing 5% FBS by centrifugation, washed twice in PBS and resuspended to a concentration of 1×10⁸ cells/ml in Tris-HCl 50 mM, KCl 300 mM, EDTA-N₂ 10 mM pH 7.4 (4° C.). Cell suspensions were homogenized with an UltraTurrax 30 s 12.000 rpm. The homogenates were centrifuged at 38.000×g (4° C.) and the pellet resuspended in Tris-HCl 50 mM pH 7.4. The homogenization was repeated once and the homogenates were incubated on ice for 45 min.

The homogenates were again centrifuged as described above and resuspended in Tris-HCl 50 mM pH 7.4. This centrifugation step was repeated 3 times in total. After the last centrifugation step the pellet was resuspended in Tris-HCl 50 mM and homogenized with Dual Potter, 10 strokes to a homogenous solution, an aliquot was removed for protein determination. Membranes were aliquoted and frozen at −80° C. until use.

The radioligand binding assay is performed at room temperature in 96-well microtiter plates (No-binding Surface Plates, Corning 3600) with a final assay volume of 200 μl/well in incubation buffer (50 mM Tris buffer (pH 7.4 RT) containing 0.1% BSA, 40 mg/L Bacitracin, complete EDTA-free protease inhibitor cocktail tablets 20 pills/L (Roche) and 3 mM MnCl₂). Competition binding curves were done by adding increasing amounts of the test compound. Test compounds were dissolved and serially diluted in DMSO, final DMSO concentration 1.5% in the assay. 50 μl Non labelled ZD 6021 (a non selective NK-antagonist, 10 μM final conc) was added for measurement of non-specific binding. For total binding, 50 μl of 1.5% DMSO (final conc) in incubation buffer was used. [³H-Sar,Met(O₂)-Substance P] (4 nM final conc) was used in binding experiments on hNK₁r. [³H-SR48968] (3 nM final conc.) for hNK₂r and [³H-SR142801] (3 nM final conc) for binding experiments on hNK₃r. 50 μl radioligand, 3 μl test compound diluted in DMSO and 47 μl incubation buffer were mixed with 5-10 μg cell membranes in 100 μl incubation buffer and incubated for 30 min at room temperature on a microplate shaker.

The membranes were then collected by rapid filtration on Filtermat B(Wallac), presoaked in 0.1% BSA and 0.3% Polyethyleneimine (Sigma P-3143), using a Micro 96 Harvester (Skatron Instruments, Norway). Filters were washed by the harvester with ice-cold wash buffer (5 mM Tris-HCl, pH 7.4 at 4° C., containing 3 mM MnCl₂) and dried at 50° C. for 30-60 min. Meltilex scintillator sheets were melted on to filters using a Microsealer (Wallac, Finland) and the filters were counted in a β-Liquid Scintillation Counter (1450 Microbeta, Wallac, Finland).

The Kᵢ value for the unlabeled ligand was calculated using the Cheng-Prusoff equation (Biochem. Pharmacol. 22:3099-3108, 1973): where L is the concentration of the radioactive ligand used and K_{d} is the affinity of the radioactive ligand for the receptor, determined by saturation binding.

Data was fitted to a four-parameter equation using Excel Fit.

Kᵢ=IC₅₀/(1+(L/K_{d}))

### Results

In general, the compounds of the invention, which were tested, demonstrated statistically significant antagonistic activity at the NK₁ receptor within the range of 8-9 for the pK_{B}. For the NK₂ receptor the range for the pK_{B} was 7-9. In general, the antagonistic activity at the NK₃ receptor was 7-8 for the pK_{B}.

In general, the compounds of the invention, which were tested, demonstrated statistically significant CYP3A4 inhibition at a low level. The IC₅₀ values tested according to Bapiro et al; Drug Metab. Dispos. 29, 30-35 (2001) were generally greater than 50 μM.

### Activity Against hERG

The activity of compounds according to formula I against the hERG-encoded potassium channel can be determined according to Kiss L, et al. Assay Drug Dev Technol. 1 (2003), 127-35: “High throughput ion-channel pharmacology: planar-array-based voltage clamp”.

In general, the compounds of the invention, which were tested, demonstrated statistically significant hERG activity at a low level. The IC₅₀ values tested as described above were generally greater than 8 μM.

### Metabolic Stabilty

The metabolic stability of compounds according to formula I can be determined as described below:

The rate of biotransformation can be measured as either metabolite(s) formation or the rate of disappearance of the parent compound. The experimental design involves incubation of low concentrations of substrate (usually 1.0 μM) with liver microsomes (usually 0.5 mg/ml) and taking out aliquotes at varying time points (usually 0, 5, 10, 15, 20, 30, 40 min.). The test compound is usually dissolved in DMSO. The DMSO concentration in the incubation mixture is usually 0.1% or less since more solvent can drastically reduce the activities of some CYP450s. Incubations are done in 100 mM potassium phosphate buffer, pH 7.4 and at 37° C. Acetonitrile or methanol is used to stop the reaction. The parent compound is analysed by HPLC-MS. From the calculated half-life, t_{1/2}, the intrinsic clearance, Clint, is estimated by taking microsomal protein concentration and liver weight into account.

In general, the compounds of the invention had in vitro metabolic stability at a high level. Intrinsic clearance values tested as above were generally lower than 40 μl/min/mg protein.

The following table illustrates the properties of the compounds of the present invention:

### 3-Chloro-N-[(2S)-2-(4-fluorophenyl)-4-(3-morpholin-4-ylazetidin-1-yl)butyl]-N-methyl-5-(trifluoromethyl)benzamide (Ex1):

| pKB | pKB | pKB | IC₅₀ | IC₅₀ | CLint |
|---|---|---|---|---|---|
| (NK1) | (NK2) | (NK3) | (hERG) | (CYP3A4) | (HLM) |
| 7.6 | 7.3 | 6.9 | 8.5 μM | >50 μM | 39 μL/min/mg |

### Biological Evalution

### Gerbil Foot Tap (NK1 Specific Test Model)

Male Mongolian gerbils (60-80 g) are purchased from Charles River, Germany. On arrival, they are housed in groups of ten, with food and water ad libitum in temperature and humidity-controlled holding rooms. The animals are allowed at least 7 days to acclimatize to the housing conditions before experiments. Each animal is used only once and euthanized immediately after the experiment by heart punctuation or a lethal overdose of penthobarbital sodium.

Gerbils are anaesthetized with isoflurane. Potential CNS-permeable NK1 receptor antagonists are administered intraperitoneally, intravenously or subcutaneously. The compounds are given at various time points (typically 30-120 minutes) prior to stimulation with agonist.

The gerbils are lightly anaesthetized using isofluorane and a small incision is made in the skin over bregma. 10 pmol of ASMSP, a selective NK1 receptor agonist, is administered icv in a volume of 5 μl using a Hamilton syringe with a needle 4 mm long. The wound is clamped shut and the animal is placed in a small plastic cage and allowed to wake up. The cage is placed on a piece of plastic tubing filled with water and connected to a computer via a pressure transducer. The number of hind feet taps is recorded.

### Fecal Pellet Output (NK2 Specific Test Model)

The in vivo effect (NK2) of the compounds of formula I can be determined by measuring NK2 receptor agonist-induced fecal pellet output using gerbil as described in e.g. The Journal of Pharmacology and Experimental Therapeutics (2001), pp. 559-564.

### Colorectal Distension Model

Colorectal distension (CRD) in gerbils is performed as previously described in rats and mice (Tammpere A, Brusberg M, Axenborg J, Hirsch I, Larsson H, Lindström E. Evaluation of pseudo-affective responses to noxious colorectal distension in rats by manometric recordings. Pain 2005; 116: 220-226; Arvidsson S, Larsson M, Larsson H, Lindström E, Martinez V. Assessment of visceral pain-related pseudo-affective responses to colorectal distension in mice by intracolonic manometric recordings. J Pain 2006; 7: 108-118) with slight modifications. Briefly, gerbils are habituated to Bollmann cages 30-60 min per day for three consecutive days prior to experiments to reduce motion artefacts due to restraint stress. A 2 cm polyethylene balloon (made in-house) with connecting catheter is inserted in the distal colon, 2 cm from the base of the balloon to the anus, during light isoflurane anaesthesia (Forene®, Abbott Scandinavia AB, Solna, Sweden). The catheter is fixed to the tail with tape. The balloons are connected to pressure transducers (P-602, CFM-k33, 100 mmHg, Bronkhorst HI-TEC, Veenendal, The Netherlands). Gerbils are allowed to recover from sedation in the Bollmann cages for at least 15 min before the start of experiments.

A customized barostat (AstraZeneca, Mölndal, Sweden) is used to manage air inflation and balloon pressure control. A customized computer software (PharmLab on-line 4.0) running on a standard computer is used to control the barostat and to perform data collection. The distension paradigm used consists of 12 repeated phasic distensions at 80 mmHg, with a pulse duration of 30 sec at 5 min intervals. Compounds or their respective vehicle are administered as intraperitoneal (i.p.) injections before the CRD paradigm. Each gerbil receives both vehicle and compound on different occasions with at least two days between experiments. Hence, each gerbil serves as its own vehicle control.

The analog input channels are sampled with individual sampling rates, and digital filtering is performed on the signals. The balloon pressure signals are sampled at 50 samples/s. A highpass filter at 1 Hz is used to separate the contraction-induced pressure changes from the slow varying pressure generated by the barostat. A resistance in the airflow between the pressure generator and the pressure transducer further enhances the pressure variations induced by abdominal contractions of the animal. A customized computer software (PharmLab off-line 4.0) is used to quantify the magnitude of highpass-filtered balloon pressure signals. The average rectified value (ARV) of the highpass-filtered balloon pressure signals is calculated for 30 s before the pulse (i.e baseline reponse) and for the duration of the pulse. When calculating the magnitude of the highpass-filtered balloon pressure signals, the first and last seconds of each pulse are excluded since these reflect artifact signals produced by the barostat during inflation and deflation and do not originate from the animal.

1. A compound of formula (I) 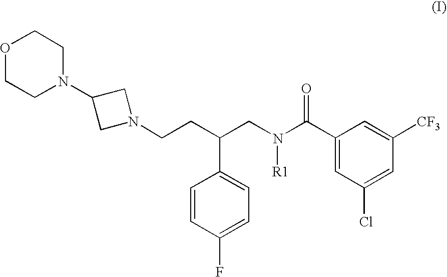
an enantiomer thereof or a pharmaceutically acceptable salt of the compound or enantiomer.
wherein
R1 is C₁-C₄ alkyl, wherein the alkyl group may be substituted by one or more fluoro atoms;

2. The compound according to claim 1, wherein R1 is methyl.

3. The compound according to claim 1, wherein R1 is ethyl.

4. The compound according to claim 1 which is 3-Chloro-N-[(2S)-2-(4-fluorophenyl)-4-(3-morpholin-4-ylazetidin-1-yl)butyl]-N-methyl-5-(trifluoromethyl)benzamide.

5. The compound according to any one of claims 1-4 wherein the compound is the (S)-enantiomer.

6. (canceled)

7. A method for the treatment of a functional gastrointestinal disorder which comprises administering to a patieent in need thereof a therapeutically effective amount of a compound according to any one of claims 1-4.

8. A method for the treatment of IBS which comprises administering to a patient in need thereof a therapeutically effective amount of a compound according to any one of claims 1-4.

9. A method for the treatment of functional dyspepsia which comprises administering to a patient in need thereof a therapeutically effective amount of a compound according to any one of claims 1-4.

10. A pharmaceutical formulation comprising a compound according to claim 1 as active ingredient and a pharmaceutically acceptable carrier or diluent.

11. A compound selected from 3-Chloro-N-[(2S)-2-(4-fluorophenyl)-4-hydroxybutyl]-N-methyl-5-(trifluoromethyl)benzamide and 3-Chloro-N-[(2S)-2-(4-fluorophenyl)-4-oxobutyl]-N-methyl-5-(trifluoromethyl)benzamide.

12. The method according to claim 7, wherein the compound is the (S)-enantiomer.

13. The method according to claim 8, wherein the compound is the (S)-enantiomer.

14. The method according to claim 9. wherein the compound is the (S)-enantiomer.

15. A method for antagonizing tachykinin action at the NIC (neurokinin) receptors in a patient, which comprises administering to the patient a therapeutically effective amount of a compound according to any one of claims 1-4.

16. The method according to claim 15, wherein the compound is the (S)-enantioiner.
